(19) United States
(12) Patent Application Publication
(10) Pub. No.: US 2018/0180586 A1
(43) Pub. Date: Jun. 28, 2018

(54) ZERO EMISSION WOBBE ANALYZER

(71) Applicant: COSA Xentaur Corporation, Yaphank, NY (US)

(72) Inventor: Bryan Flanagan, Shirley, NY (US)

(21) Appl. No.: 15/852,659

(22) Filed: Dec. 22, 2017

Related U.S. Application Data
(60) Provisional application No. 62/438,214, filed on Dec. 22, 2016.

(51) Int. Cl. G01N 33/22

(52) U.S. Cl. CPC G01N 33/225

(57) ABSTRACT

Apparatuses and methods for measuring characteristics of a gaseous fuel are provided. For example, an apparatus comprises injection valves controllable at a preset frequency and duty cycle. A first injection valve is connected to an air supply line and a first replaceable orifice. A second replaceable orifice is connected to a common supply line. The common supply line is selectively coupled, via a valve system, to one of a plurality of calibration gases or at least one gas under testing. The air and one of the calibration gases or one of the gases under testing are supplied to a mixer through the respective injection valves and the respective orifices. The mixer outputs an output gas via an output gas line to a heating element. The preset frequency, duty cycle and dimensions of the output gas line are set to have a continuous gas flow to the heating element.

### CROSS-REFERENCE TO RELATED APPLICATIONS

This application claims the benefit of and priority to U.S. Provisional Application Ser. No. 62/438,214 filed Dec. 22, 2016, the contents of which is incorporated by reference herein.

### FIELD OF THE DISCLOSURE

This disclosure relates to an apparatus, system and method for measuring characteristics of gaseous fuel including WOBBE index.

### BACKGROUND

A WOBBE index is an indicator for combustion energy of different compositions of gases for a given pressure.

One way of measuring a WOBBE index is to use a residual oxygen method and a fast loop to exhaust excessive air/gas mixtures that are not burnt.

However, the mixture in the fast loop is required to be disposed of.

### SUMMARY

Disclosed is an apparatus for measuring a WOBBE Index such as a calorimeter. The apparatus comprises an air supply line for supplying air, a first replaceable orifice connected to the air supply line , a process gas supply line for supplying gas under testing; a plurality of calibration gas supply lines, each respectively supplying a different gas for calibration, a valve system coupled to the plurality of calibration gas supply lines and the process gas supply line for selectively supply the gas under testing or one of the different gases for calibration to a common supply line, a second replaceable orifice connected to the common supply line and a mixer having a first input and a second input and a gas output. Air from the first replaceable orifice is supplied to the first input and gas for calibration or gas under testing from the second replaceable orifice is supplied to the second input. The apparatus further comprises a first injection valve connecting the air supply line and the first replaceable orifice and configured to inject air into the first replaceable orifice, and a second injection valve connecting the common supply line and the second replaceable orifice and configured to inject gas for calibration or gas under testing into the second replaceable orifice. The apparatus further comprises an output gas line connected to the gas output. The output gas line is also connected to a heating element. The apparatus further comprises a sensor disposed near the heating element configured to sensor property of output of the heating element and providing a signal indicating of the property. The apparatus further comprises a first controller configured to control the first injection valve at a preset frequency and a duty cycle and a second controller configured to control the second injection valve at the preset frequency and the duty cycle. The preset frequency, the duty cycle and dimensions of the output gas line are set to have a continuous gas flow into the heating element. The signal from the sensor is used to determine a measure of a WOBBE index of the gas under testing.

In other aspects, the apparatus may comprises a plurality of process gas supply lines for supplying different gases under testing and the valve system comprises respective valves controllable to selectively supply one of the different gases under testing to the common supply line. The apparatus further comprises a valve controller, the valve controller configured to open or close the respective valve in the valve system to selectively supply one of the different gases under testing to the common supply line under control of the processor.

In other aspects, the apparatus is configured to communicate with a distributed control system and receive an instruction from the distributed control system to cause one of the different gases under testing to be supplied to the common supply line.

In other aspects, the first replaceable orifice and the second replaceable orifice are a first set of replaceable orifices and the apparatus further comprises a second set of replaceable orifices.

The second set of replaceable orifices comprises a third replaceable orifice and a fourth replaceable orifice. The third replaceable orifice is connected to the air supply line and a first switchover valve. The first switchover valve is configured to switch the air flow towards one of the first replaceable orifice and the third replaceable orifice. The fourth replaceable orifice is connected to the common supply line and a second switchover valve. The second switchover valve switching a flow of the gas under testing or one of the different gases for calibration towards one of the second replaceable orifice and the fourth replaceable orifice.

The second set of replaceable orifices is configured for use for gases having higher energy than the first set of replaceable orifices.

The processor is configured to determine which set of replaceable orifices to use for monitoring of the gas under testing based on a comparison of level indicated by a signal output from the sensor for the gas under testing and one or more preset thresholds.

### BRIEF DESCRIPTION OF THE FIGURES

FIG. 1 depicts a block diagram of an apparatus in accordance with aspects of the disclosure;

FIG. 2 depicts a valve system on each of the sample and calibration gas lines in accordance with aspects of the disclosure;

FIG. 3 depicts a flow chart for setting the frequency and duty cycle for the injection valves in accordance with aspects of the disclosure;

FIG. 4 depicts a flow chart for calibrating the apparatus in accordance with aspects of the disclosure;

FIG. 5 depicts a block diagram of another apparatus in accordance with aspects of the disclosure;

FIG. 6 depicts a flow chart for switching sample streams and determining a wobbe index for a sample stream in accordance with aspects of the disclosure;

FIG. 7 depicts a flow chart for switching samples streams and determining a wobbe index for a sample stream in accordance with other aspects of the disclosure;

FIG. 8 depicts a block diagram of another apparatus in accordance with aspects of the disclosure;

FIGS. 9A and 9B depict a flow chart for calibrating the another apparatus in accordance with aspects of the disclosure; and

FIG. 10 depicts a flow chart for determining a wobbe index in accordance with other aspects of the disclosure.

### DETAIL DESCRIPTION

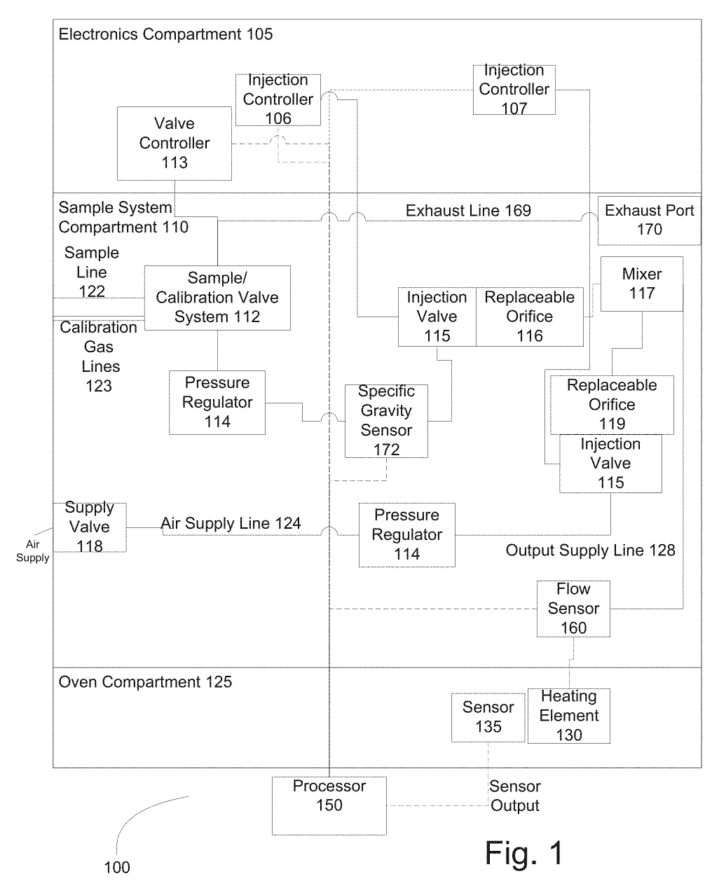

FIG. 1 shows an apparatus 100 for measuring a WOBBE index in accordance with aspects of the disclosure.

The apparatus 100 includes a three part casing. The three part casing includes an electronics compartment 105, sample system compartment 110 and an oven compartment 125. The electronics compartment 105 houses a valve controller 113 and injection controllers 106, 107, which will be described later in detail. Additionally, in an aspect of the disclosure, the electronics compartment 105 can also house the Processor 150. The Processor 150 is configured to control the valve controller 113, the injection controllers 106, 107 and calculate the WOBBE Index based on a sensor 135 output and the specific gravity sensor 172.

The sample system compartment 110 houses the components that transfer the sample/calibration gases and air to the oven, e.g., heating element 130. The gases, whether sample, calibration gases or air, enter the compartment via compression type fittings such as bulkhead fitting.

In accordance with aspects of the disclosure, the apparatus 100 may be used to determine a Wobbe index for at least one sample fuel. The sample fuel enters the apparatus via the above-identified compression type fitting and travels through the sample line 122. When more than one sample fuel is coupled to the apparatus (as will be described later), each sample enters the apparatus via a respective compression type fitting and a respective sample line 122. Flow of the sample fuel is controlled via the Sample/Calibration Valve system 112, which will be described later in detail.

Similarly, calibration gases enter the apparatus 100 via the above-identified compression type fitting and travel through respective calibration gas lines 123 (also referenced as calibration line or a respective calibration gas line). In accordance with aspects of the disclosure, two or more calibration gases may be used to calibrate the apparatus. In FIG. 1, there are two calibration gases, e.g., a low calibration gas and a high calibration gas. FIG. 1 shows two calibration gas lines. Each gas enters the apparatus 100 via its respective compression type fitting and travel through a respective calibration gas line. Flow of the respective calibration gases is controlled via the Sample/Calibration Valve system 112.

The Sample/Calibration Valve system 112 includes three sets of supply valves: one set for the sample gas (also referenced as a process gas or gas under testing), one set for a low calibration gas and another set for a high calibration gas.

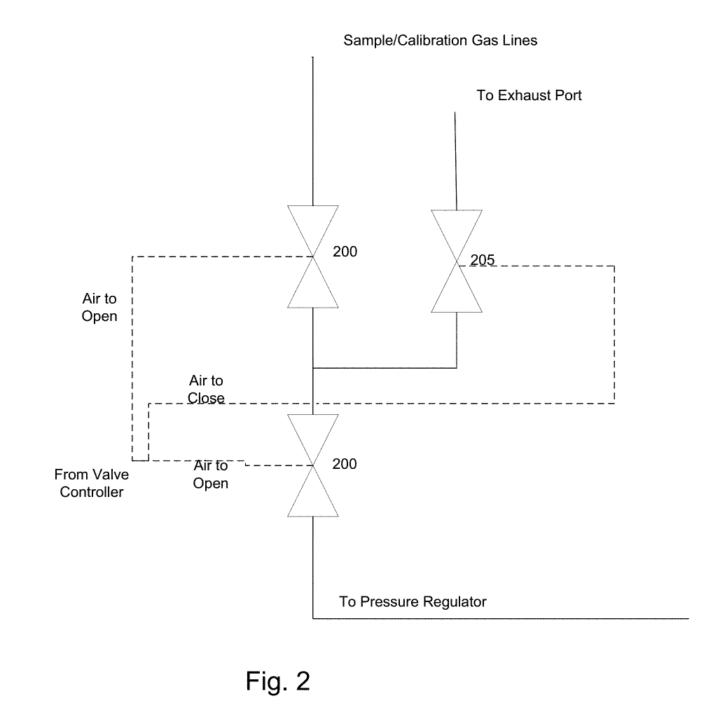

FIG. 2 illustrates one of the sets of supply valves in accordance with aspects of the disclosure. Each line includes a similar set of supply valves. As shown in FIG. 2, each line includes a pair of valves 200. The pair of valves 200 are connected in series. Additionally, each line also includes a valve 205 connected between the pair of valves 200 on one end and connects to an exhaust line 169 and to an exhaust port 170 on the other end. The pair of valves 200 are normally closed and the valve 205 is normally open. In other words, when the sample or calibration gas is not controlled to flow, the valves 200 are closed, whereas the exhaust valve 205 is opened which allows the gas to flow through the exhaust line 169 to the exhaust port 170 and out of the apparatus 100. In an aspect of the disclosure, the valves may be gate valves. In an aspect of the disclosure, the valves are pneumatically controlled. Valves 200 are supply air to open the valve and valve 205 (exhaust) is supplied air to close the valve 205. The valve controller 113 (in conjunction with processor 150) controls the air supply to the respective valves.

Air also enters the apparatus via the compression type fitting then through the air supply valve 118. In an aspect of the disclosure, air is supplied to the valve controller 113 and injection controller 106, 107.

A pressure regulator 114 is disposed downstream of the Sample/Calibration Valve System 112 on the sample/calibration line (also references as a common line or sample calibration gas line). A similar pressure regulator 114 is disposed on the air supply line 124. In an aspect of the disclosure, the air supply line 124 has a diameter greater than the diameter of the sample line 122 or calibration gas lines 123 (sample/calibration gases line).

In an aspect of the disclosure, the apparatus 100 further comprises a specific gravity sensor/detector 172. In an aspect of the disclosure, the specific gravity sensor/detector 172 oscillates based on the specific gravity of the gas. The oscillation is calibrated in advance. In an aspect of the disclosure, the specific gravity sensor/detector 172 may detect a specific gravity between 0-3. In aspect of the disclosure, the specific gravity is measured by reference to a known frequency. The gas passes through a chamber that comprising an oscillating spool. A base frequency (known) and changes with the specific gravity of the gas. The change is oscillation is used to determine the specific gravity.

Injection valves 115 are disposed on the air supply line (air line) and the sample line/calibration line downstream of the pressure regulators 114.

The apparatus 100 also comprises at least one set of replaceable orifices 116/119. One replaceable orifice is used for the sample/calibration gas 116 and another replaceable orifice 119 is used for the air.

The apparatus 100 also comprises a mixer 117. The sample gas/calibration gas and air is mixed in the mixer 117. The phrase sample line refers to a pathway between the compression type fitting and the mixer for the sample. The phrase calibration gas line 123 refers to pathways between the respective compression type fitting and the mixer for each calibration gas. The phrase sample/calibration gas line refers to the joint pathway for the sample or the calibration gas between the valve system 112 and the mixer 117. Between the valve system 112 and the mixer 117 the pathways are the same.

In accordance with aspects of the disclosure, the injection values 115 are controlled by the respective injection controller 106, 107 to inject gas and air (at the same time) into the mixer 117. The quantities of the gas and air can be controlled through the use of the replaceable orifices 116, 119, respectively. The size of the orifices can be selected based on the energy of the gas under testing. In an aspect of the disclosure, the size of the orifice for the air (119) is larger than the size of the orifice for the gas under testing (116). For example, a 0.2 mm gas orifice and a 0.36 mm air orifice can be used. In another aspect of the disclosure a 0.1 mm gas orifice and a 0.56 mm air orifice can be used.

However, the relative size and dimensions can be changed. In another aspect of the disclosure, the size of the orifice may be selected based on an expected energy of the sample.

In an aspect of the disclosure, the mixer 117 is a static mixer.

The sample and calibration gases are supplied to the apparatus 100 under a known pressure range. For example, the pressure range can be 35-80 psig.

The Processor 150 is configured to control the respective supply valves 200 and exhaust valve 205 as needed to supply either the sample (process gas) or calibration gases (low/high).

The respective pressure regulators 114, one on each of the air supply and gas lines is used to provide a known, constant gas/air flow pressure to the injection valves 115. For example, the pressure regulators can be set between 0 and 80 psig. In an aspect of the disclosure, the pressure on the gas/sample line can be set to the same as the pressure in the air flow line. In another aspect of the disclosure, the pressure can be set to a different value. For example, the pressure for the gas/sample line can be set to 30 psig and 27 psig for the air supply.

The Processor 150 is configured to control the injection valves 115 using the respective injection controllers 106, 107 to inject gas and air (at the same time) into the mixer 117 using a set frequency and duty cycle. In an aspect of the disclosure, the injection controllers 106, 107 are solenoids. While FIG. 1 depicts the injection controller 106, 107 separately, the controller 106, 107 may be in a single device. The Processor 150 supplies pulse signals to the injection controllers 106, 107.

The frequency and duty cycle can be varied. For example, a frequency of 500 mHz and a duty cycle of 40% can be used. In another aspect of the disclosure, a frequency of 90 mHz and a duty cycle of 2% can be used. In yet another aspect of the disclosure, a frequency of 0.8 Hz and a duty cycle of 40% can be used. The duty cycle and the frequency may be set to a specific value during a calibration process.

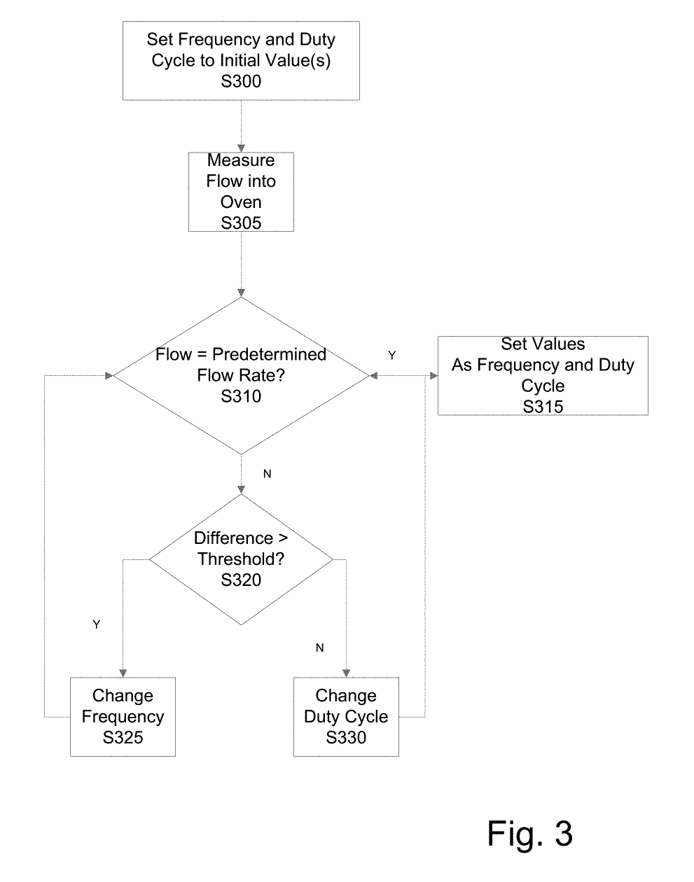

FIG. 3 illustrates an example of a calibration process in accordance with aspects of the disclosure. The frequency and duty cycle is set such that a continuous predetermined flow rate of supplied to the heat element 130. For example, a continuous predetermined flow rate may be 800 CCM. However, the flow rate described herein is just an example of a predetermined flow rate and other flow rates may be used in accordance with aspects of the disclosure. A flow sensor 160 is disposed between the mixer 117 and the heating element 130 and is configured to detect the flow rate. The flow sensor 160 is in communication with the processor 150. At S300, the processor 150 sets the frequency and duty cycle to initial default values (one for the frequency and one or the duty cycle). In an aspect of the disclosure, the processor 150 then causes air to be supplied via the air supply line and a gas to be supplied through a calibration gas line (not shown in FIG. 3). Any gas may be used to calibrate the frequency and duty cycle irrespective of its Wobbe index value. The processor 150 supplies pulse signals to the valve controller 113 to cause the valves 200 to actuate for the corresponding calibration gas line. At S300, the processor controls the injection valves 115 using the set initial default values. At S305, the processor receives a signal from the flow sensor 160. A signal from the flow sensor is shown in FIG. 1 by a dashed line. The received flow rate is compared with the predetermined flow rate, e.g., target, at S310. If the sensed flow rate equals the predetermined flow rate, the initial default values are set as the frequency and duty cycle for subsequent use at S315, otherwise, the magnitude of the difference is determined. The frequency is used as a coarse change whereas the duty cycle is used for a fine change of the flow rate. In an aspect of the disclosure, the difference between the sensed and predetermined flow rates is compared with a threshold at S320. A large difference indicates that the frequency should be changed (S325) whereas a small difference indicates that the duty cycle should be changed (S330). The process is repeated until the sensed flow rate equals the predetermined flow rate. For example, if the sensed flow rate is much smaller than the predetermined flow rate (based on the threshold comparison), the frequency is increased. In another example, if the sensed flow rate is slightly smaller than the predetermined flow rate (based on the threshold comparison), the duty cycle is increased.

The Processor 150 controls the injection valves 115 to inject the air/gas at the same time into the mixer 117. The mixer 117 mixes the gas and air and outputs the mixture to the heating element 130 via an output supply line 128. The output supply line 128 has a preset diameter. In an aspect of the disclosure, the output supply line 128 is tapered as the flow moves downstream. For example, in an aspect of the disclosure, the diameter of the output supply line changes from 0.25 inches to 0.125 inches. In another aspect of the disclosure, the output supply line further comprises a capillary tube. In an aspect of the disclosure, the capillary tube may have an internal diameter of 0.02″. The tube size is chosen to five a steady flow toward the heating element 130, such as a oven, at a constant rate. Additionally, the tube size provides a limit to the flow rate, for safety. The diameter of the capillary tube described herein is just an example of a diameter for the tube and other diameters may be used in accordance with aspects of the disclosure to provide a steady and constant flow rate. The heating element 130 burns all of the air/gas supplied thereto.

In another aspect of the disclosure, instead of Processor 150 supplying the signal to the respective injection controller 106, 107, and valve controller 113, a separate frequency generator can be used to supply the same.

While the air/gas mixture is heated by the heating element, a sensor 135, such as an oxygen sensor, detects the residual oxygen level given off as the air/gas is burnt. In an aspect of the disclosure, the oxygen sensor is a zirconia oxide cell. The Sensor 135 supplies the detection result, e.g., sensor output, to the Processor 150. The Processor 150 calculates the WOBBE index using the output from the sensor.

The WOBBE index=Heating Value/√Specific Gravity (1)

Although, the gas and air are supplied to the mixer 117 via injection values, the frequency and duty cycle is selected in combination with the diameter of the output supply line 128 such that a continuous supply of mixed gas/air is processed by the heating element 130, e.g., in a manner as described above. Advantageously, this configuration reduces the amount of gas/air mixture needed to perform the measurement from prior systems. Additionally, the apparatus eliminates the need for the fast loop.

Moreover, since continuous supply of mixed gas/air is processed by the heating element, even though injection valves are used, the apparatus avoids spikes in the WOBBE measurement.

As described above, there are two calibration gases, one having a known low WOBBE index and one having a known high WOBBE index or a known low BTU and high BTU. For example, in an aspect of the disclosure CH₄ (25%) and N₂ (75%) can be used as the low and CH₄ (100%) can be used as the high. The respective WOBBE index values for the same are 244.7 and 1223.3.

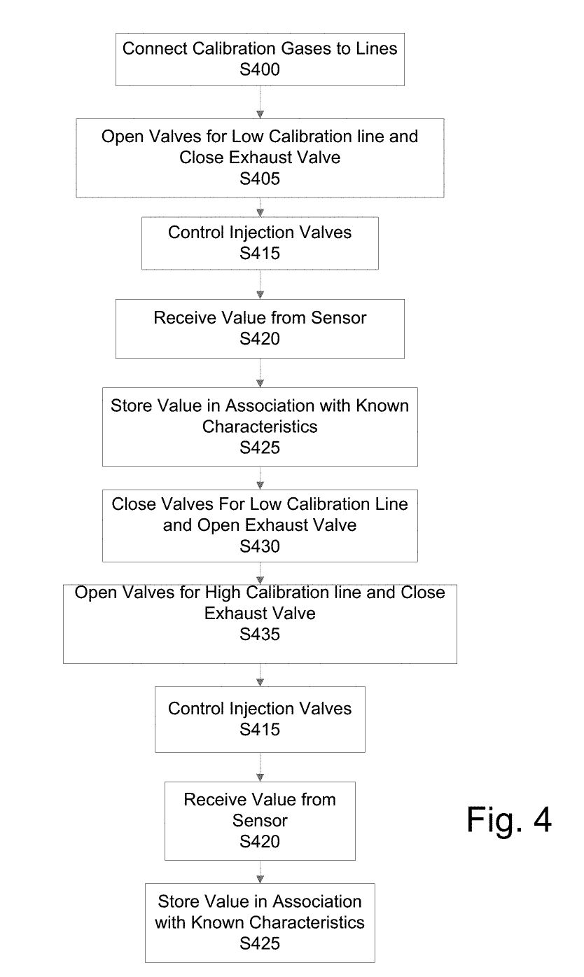

FIG. 4 illustrates a flow chart for a method of calibrating the apparatus in accordance with aspects of the disclosure using the two calibration gases. At S400, each calibration gas is connected to a respective calibration gas line. In an aspect of the disclosure, a user couples an external pipe to the compression type fitting for each calibration gas line. Once attached, the calibration gas is in communication with a respective calibration gas line 123. At S405, the processor 150 controls the valves 200 to open (for the low calibration gas line) and the valve 205 to close for the exhaust line 169. In an aspect of the disclosure, the processor 150 transmits a pulse signal to the valve controller 113. The valve controller 113 controls the valves 200/205 using air to open or close the respective valves. Once the valves 200 are open and the valve 205 is closed, the calibration gas (low) is supplied to the sample/calibration gas line. At this time, the supply valve 118 is also open allowing the air supply to enter the air supply line 124. If calibrating using a WOBBE index verses a BTU value, the specific gravity of the calibration gas (is determined) using the specific gravity sensor 172. At S415, the processor 150 controls the injection valves 115 using the set frequency and duty cycle. In an aspect of the disclosure, the processor 150 transmits a control pulse as described above to the injection controllers 106, 107 and the injection controllers control the respective injection valves 115.

At S420, the processor 150 receives a signal from the sensor 135 indicative of the residual oxygen resulting from heating using the heating element 130. In an aspect of the disclosure, the signal indicates a voltage level corresponding to the residual oxygen. At S425, the processor 150 stores the voltage level in a storage device, such as memory in association with the known characteristics of the low reference gas, e.g., BTU. The processor 150 may also calculate the Wobbe Index value using equation 1.

Thereafter, the processor 150 controls the valves 200 to close (for the low calibration gas line) and the valve 205 to open for the exhaust line 169 at S430. In an aspect of the disclosure, the processor 150 transmits a pulse signal to the valve controller 113. The valve controller 113 controls the valves 200/205 using air to close or open the respective valves. Once the valves 200 are closed and the valve 205 is open, the calibration gas (low) is isolated from the sample/calibration gas line downstream of the valve system 112. The calibration gas may vent via the exhaust line 169 through the exhaust port 170.

The process is repeated for the high calibration gas. At S435, the processor 150 controls the valves 200 to open (for the high calibration gas line) and the valve 205 to close for the exhaust line 169. In an aspect of the disclosure, the processor 150 transmits a pulse signal to the valve controller 113. The valve controller 113 controls the valves 200/205 using air to open or close the respective valves. Once the valves 200 are open and the valve 205 is closed, the calibration gas (high) is supplied to the sample/calibration gas line. At this time, the supply valve 118 is also open allowing the air supply to enter the air supply line 124. S415-S435 are repeated for the high calibration gas. Once the low/high voltages are stored, the processor can subsequently determine a BTU value for any sample gas via interpolation. In an aspect of the disclosure, the relationship is linear, e.g., a line connecting the two calibration points may be used for the interpolation. Afterwards, the Wobbe index may be calculated from the BTU value and the specific gravity value.

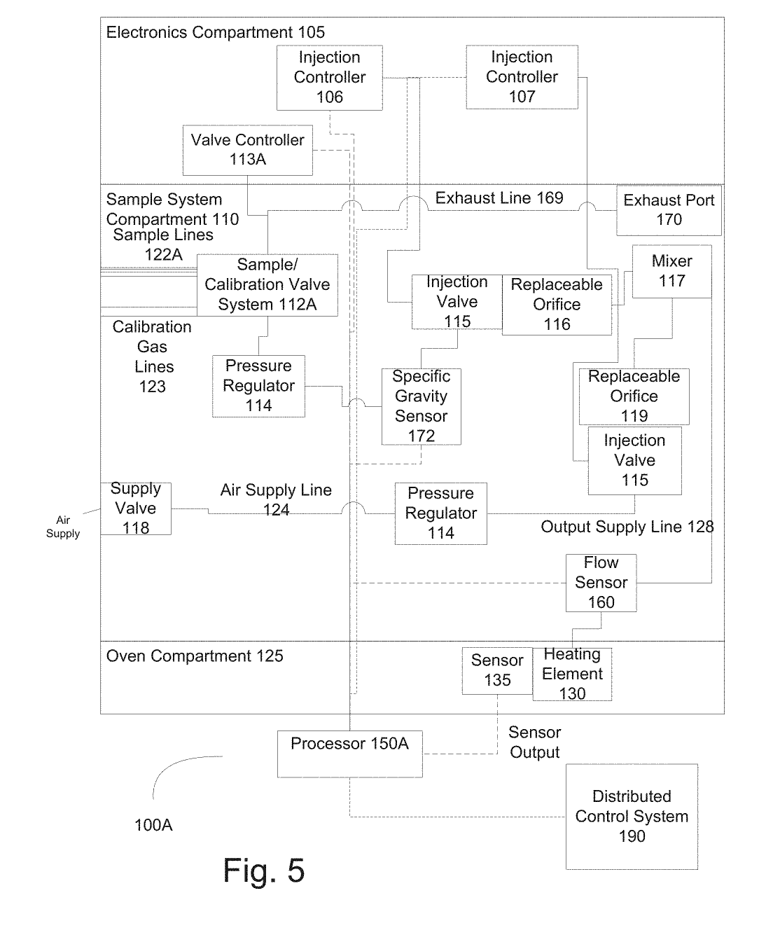

FIG. 5 illustrates another apparatus 100A in accordance with aspects of the disclosure. In apparatus 100A a plurality of samples may be coupled to the apparatus via different sample lines 122A. FIG. 5 shows four sample lines 122A. However, the disclosure is not limited to four sample lines. Any number of sample lines may be used as needed. In apparatus 100, one sample may be coupled via the compression type fitting to the sample line; however, the apparatus 100A has a plurality of compression type fitting on the housing for samples and a plurality of sample lines. For example, the apparatus 100A may have two, three, four or more compression type fittings on the housing an respective sample lines 122A. The sample/calibration valve system 112A in apparatus 100A includes an additional set of valves 200/2005 for each additional sample line. For example, when the apparatus 100A includes four sample lines, the sample/calibration valve system 112A comprises four sets of valves 200/205 for the sample lines. Additionally, similar to the sample/calibration valve system 112, the sample/calibration valve system 112A may comprise two sets of valves 200/205 for the two calibration gas lines 123. The valve controller 113A is similar to valve controller 113, but has additional control elements for the additional sets of valves, e.g., additional solenoids.

In accordance with aspects of the disclosure, the processor 150A may communicate with a distributed control system (DCS) 190. A DCS is a control system, typically processor-based used in a process or plant, typically with a large number of control loops, where autonomous controllers are distributed throughout the system. The DCS acts as a central operator supervisory control. A DCS increases reliability and reduces installation cost by localizing control functions near the process plant with remote monitoring and supervision. The communication may be wireless communication using any wireless protocol (via a wireless interface, not shown in FIG. 5). In another aspect of the disclosure, the processor 150A may be connected to the DCS 190 using a wire. The remaining components of apparatus 100A are similar as apparatus 100 and will not be described again in detail.

In an aspect of the disclosure, switching of the samples for monitoring may be triggered from the DCS 190.

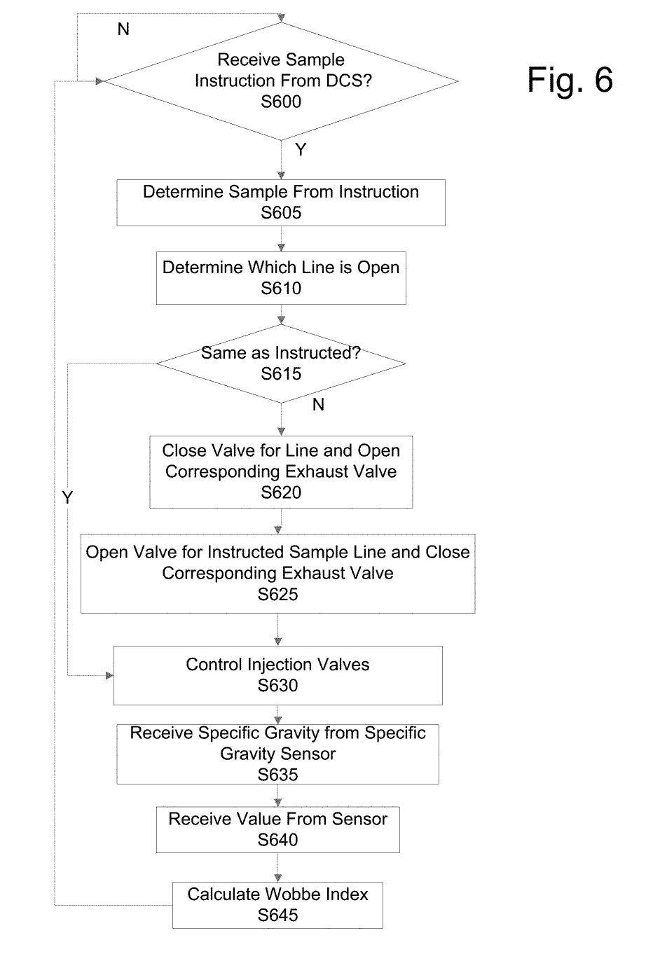

FIG. 6 depicts a flow chart for switching sample streams and determining a wobbe index for a sample stream in accordance with aspects of the disclosure. At S600, the processor 150A determines if an instruction has been received from the DCS 190 to monitor one of the plurality of sample lines. In an aspect of the disclosure, each sample line is assigned a unique identifier and the instruction may include the unique identifier. The unique identifier may be the line number such as 1-N. In another aspect of the disclosure, the unique identifier is related to the monitor gas (sample). The unique identifier is prestored in a data storage device such as memory. The instruction may include a preset header or identifier. Thus, in an aspect of the disclosure, the determination is S600 is whether a signal or instruction from the DCS 190 contains the preset header or identifier.

If an instruction is received from the DCS at S600 (“Y” at S600), at S605, the processor 150A determines which sample line should be coupled to the mixer 117. In an aspect of the disclosure, the processor 150A compares the identifier received in the instruction with the unique identifiers stored in the storage device, e.g., a match indicates a target sample line.

At S610, the processor 150A determines what line is currently open to the mixer. In an aspect of the disclosure, one line is always open, e.g., one of the plurality of sample lines 122A or one of the calibration gas lines 123. Additionally, in an aspect of the disclosure, the processor 150A may maintain a history log of the switching, e.g., log of which line is connected. The history log may be purged or deleted periodically. In another aspect of the disclosure, instead on maintaining a history log in the storage device, the processor 150A may maintain a “current connected” log indicating which line is currently open.

At S615, the processor 150A compares the target sample line with the determined open line. When the lines are not the same (“N” at S615), the processor changes lines. In an aspect of the disclosure, the processor 150 controls the valves 200 to close (for the opened line) and the valve 205 to open for the exhaust line 169. In an aspect of the disclosure, the processor 150 transmits a pulse signal to the valve controller 113A. The valve controller 113A controls the valves 200/205 using air to close or open the respective valves. Once the valves 200 are closed and the valve 205 is open, the gas (of the previously opened line) is isolated from the remaining portion of the sample/calibration gas line downstream of the valve system 112. The gas may vent via the exhaust line 169 through the exhaust port 170.

At S620, the processor 150A controls the valves 200 to open (for the target sample line) and the valve 205 to close for the exhaust line 169. In an aspect of the disclosure, the processor 150A transmits a pulse signal to the valve controller 113A. The valve controller 113A controls the valves 200/205 using air to open or close the respective valves. Once the valves 200 are open and the valve 205 is closed, the sample gas is supplied to the sample/calibration gas line. At this time, the supply valve 118 is also open allowing the air supply to enter the air supply line 124. At S630, the processor 150A controls the injection valves 115 using the set frequency and duty cycle. In an aspect of the disclosure, the processor 150A transmits a control pulse as described above to the injection controllers 106, 107 and the injection controllers control the respective injection valves 115 resulting in air and the sample gas being input into the mixer 117.

At S635, the processor 150A receives a signal from the specific gravity sensor 172 indicates the specific gravity of the sample gas. S635 may occur before S630. The received specific gravity is stored in a storage device.

Once the gas/air is mixed it is supplied to the output supply line 128 and heated by the heating element 130. At S640, the processor 150A receives a signal from the sensor 135 indicating a voltage level corresponding to the residual oxygen. In an aspect of the disclosure, the voltage level may be temporally stored in the storage device. At S645, the processor 150A determines the Wobbe index. In an aspect of the disclosure, the processor 150A retrieves the stored two calibration voltage values, interpolates a BTU value for the received voltage level. The interpolated BTU value is divided by the square root of the received specific gravity value in S635. The calculated Wobbe index value may be displayed. In an aspect of the disclosure, the processor 150A may transmit the calculated Wobbe index value to the DCS. After S645, the processor 150A waits to receive another instruction from the DCS, e.g., monitor another sample gas.

In another aspect of the disclosure, instead of receiving an instruction from the DCS 190 or in addition to the same, the processor 150A may switch sample lines based on a preset time schedule. In an aspect of the disclosure, the time schedule may be programmed via the DCS 190 and transmitted to the processor 150A via the wired or wireless interface as described above. In another aspect of the disclosure, the processor 150A is coupled to a local user interface, such as a touch panel or screen, mouse or keypad (not shown in FIG. 5). In accordance with this aspect of the disclosure, the processor 150A may also include an external clock to determine the time, e.g., whether the current time equals a scheduled time. As described above, each sample line is uniquely identified, thus, the time schedule will include the unique identifier and an associated time. The time schedule may include a sequentially looping of each of the plurality of sample lines periodically. For example, the time schedule may include every morning at 8:00 A.M. sequentially switching each of the plurality of sample lines for monitoring.

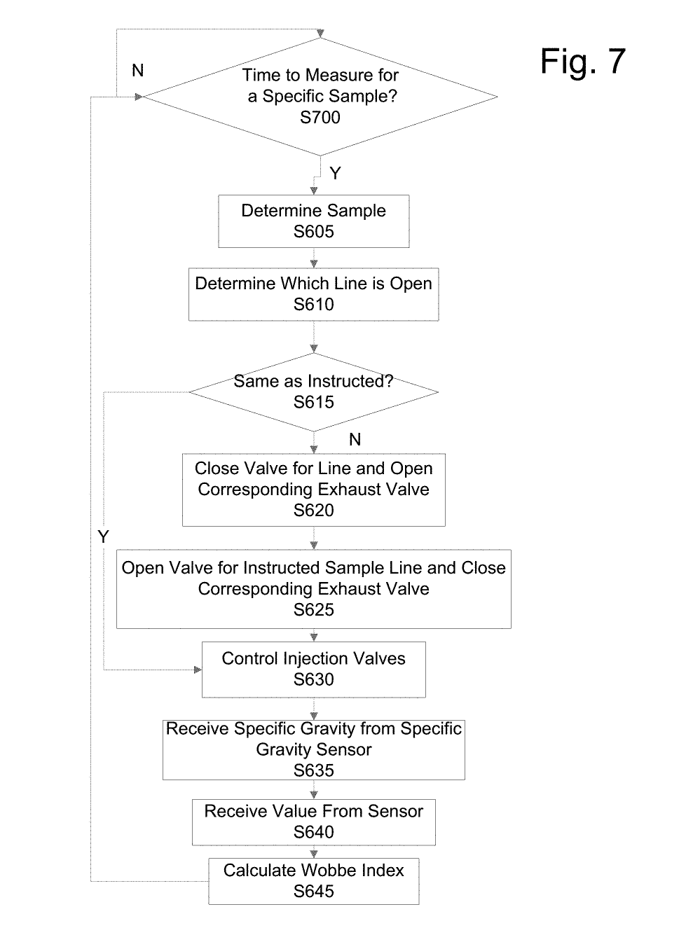

FIG. 7 depicts a flow chart for switching sample (as referenced as sample streams) in accordance with a time schedule and determining a wobbe index for the sample stream in accordance with aspects of the disclosure. The process depicted in FIG. 7 is similar to FIG. 6 except that the processing in FIG. 6 is started by the instruction from the DCS (S600) whereas the process in FIG. 7 is started with determining whether the current time is equal to a time schedule for a specific sample line (S700). In an aspect of the disclosure, the processor 150A continuously compares the current time with times in the schedule. When the current time equals a time in the time schedule, the processor 150A moves to S605, otherwise, the processor 150A loops back to S700.

As described above, both a time schedule and instruction from the DCS 190 may be used together. In an aspect of the disclosure, the instruction from the DCS 190 may have priority to a time schedule if an instruction from the DCS 190 is received when monitoring a specific sample line is being conducted or within a preset period of time upon receipt of the instruction from the DCS 190.

In another aspect of the disclosure, switching may be manually triggered by a user or operator issuing a specific local instruction via a user interface such as described above.

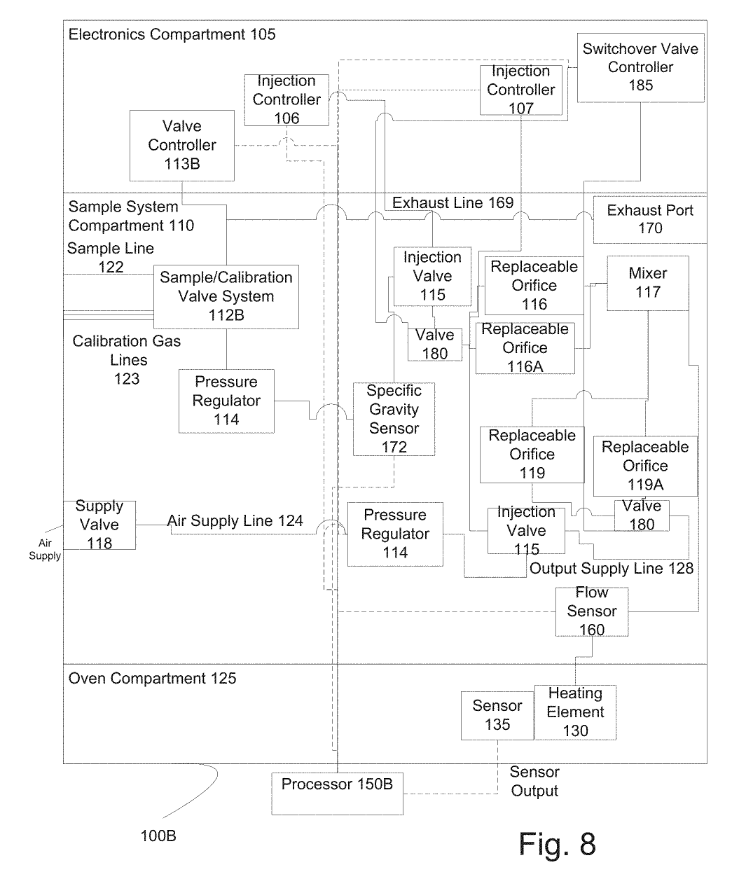

FIG. 8 illustrates another apparatus 100B in accordance with aspects of the disclosure. A difference between the apparatus 100B and apparatuses 100/100A is that apparatus 100B comprises two sets of replaceable orifices 116/119 and 116A/119A. Apparatuses 100/100A comprise one set of replaceable orifices 116/119. Having two sets of replaceable orifices allows for a greater range of sample monitoring, e.g., greater range of BTUs. A range of the sample monitoring is determined by the low and high calibration gases and the orifice diameter sizes. The higher the energy of the gas, e.g., higher BTU, the air orifice needs to be higher to allow more air in the the mixture to burn the gas. In an aspect of the disclosure, this range may be 1200 BTU. In apparatus 100B, the range can be extended. For example, the BTU range may be extended to 2800 BTU.

In an aspect of the disclosure, the second set of replaceable orifices 116A and 119A has a larger replaceable orifice 119A than the replaceable orifice 119 and a smaller replaceable orifice 116A than the replaceable orifice 116. Thus, the second set of replaceable orifices can be used for gases having higher BTUs. Flow into the sets of replaceable orifices is controlled using a pair of valves 180. In an aspect of the disclosure, each valve is a switchover valve. One switchover valve 180 is disposed between the injection valve 115 and replaceable orifices 116/116A and the other switchover valve 180 is disposed between the injection valve 115 and replaceable orifices 119/119A. The valves 180 are controlled using a switchover valve controller 185. The switchover valve controller 185 is disposed in the electronics compartment 105. In an aspect of the disclosure, the switchover valve controller 185 is a solenoid. In an aspect of the disclosure, the processor 150B issues a pulse signal to the switchover valve controller 185 to switch flows between the first set of replaceable orifices 116/119 and the second set of replaceable orifaces 116A/119A.

The apparatus 100B comprises three calibration gas lines 123 (apparatuses 100/100A comprises two). The three calibration gas lines are coupleable to a low, middle and high calibration gas. The same gas as described above may be used for the low calibration gas. The “high” calibration gas described above may be used for the middle calibration gas. For example, hexane C₆H₁₄, which has a BTU of 2663.6 may be used as the “high” calibration gas. Other gases may also be used such as, but not limited to, propane C₃H_{8.} 100% Propane has a BTU of 1899.5.

During the calibration process, two sets of calibration values are generated. A first set of calibration values for the first set of replaceable orifices 116/119 and a second set of calibration values for the second set of replaceable orifices 116A/119A. The first set of calibrations values are generated from the low and middle calibration gas and the second set of calibration values are generated from the middle calibration gas and the high calibration gas. The first set of calibration values are generated when the calibration gases flow through the first set of replaceable orifices 116/119 and the second set of calibration values are generated when the calibration gases flow through the second set of replaceable orifices 116A/119A. The middle calibration gas is used for both sets, in the first set, the middle calibration gas is a high calibration point and, in the second set, the middle gas is a low calibration point.

Sample/Calibration Valve System 112B differs from valve system 112/112B in that the valve system 112B comprises an additional set of valves (shown in FIG. 2) for the third calibration line. Valve systems 112/112B comprises two sets of valves, one for each calibration line. The valve controller 113B is similar to valve controller 113, but has additional control element for the additional set of valves, e.g., additional solenoids.

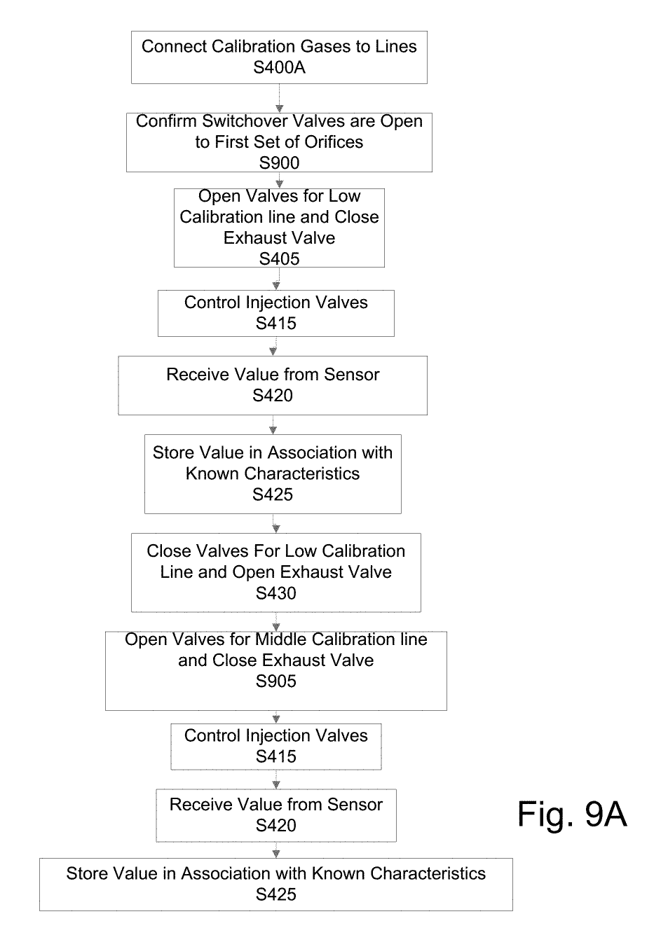

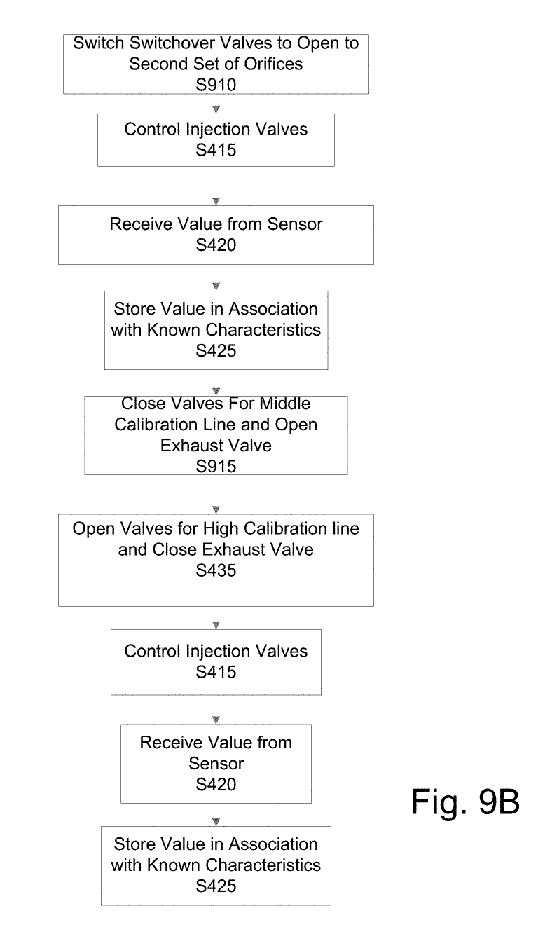

FIGS. 9A and 9B depict a flow chart for calibrating apparatus 100B in accordance with aspects of the disclosure, FIG. 9A depicts the calibration for the first set of replaceable orifices 116/119 and FIG. 9B depicts the calibration for the second set of replaceable orifices 116A/119A.At S400A, the three calibration gases are coupled to the respective lines. In an aspect of the disclosure, a user couples an external pipe to the compression type fitting for each calibration gas line. Once attached, the calibration gas is in communication with a respective calibration gas line 123. At S900, the processor 150B confirms that the valves 180 are open to the first set of replaceable orifices 116/119. In an aspect of the disclosure, the processor 150B stores a current position of the valves 180 in the data storage device. In accordance with this aspect, the processor 150B retrieves the current position to confirm the current position is open to the first set of replaceable orifices 116/119. In another aspect of the disclosure, the processor 150B instructs the switchover valve controller 185 to open the switchover valve 180 to the first set of replaceable orifices 116/119 irrespective of the current position. Once confirmed, the processor 150B executes S405-S430 to record the voltage of the low calibration point, e.g., corresponding to the low calibration gas. S405-S430 were described above and will not be described again.

Once the valves 200 for the low calibration gas line are closed (and 205 opened for the exhaust line), the processor 150B controls the valves 200/205 for the middle calibration gas line. The processor 150B controls the valves 200 to open (for the middle calibration gas line) and the valve 205 to close for the exhaust line 169. In an aspect of the disclosure, the processor 150B transmits a pulse signal to the valve controller 113B. The valve controller 113B controls the valves 200/205 using air to open or close the respective valves. Once the valves 200 are open and the valve 205 is closed, the calibration gas (middle) is supplied to the sample/calibration gas line. At this time, the supply valve 118 is also open allowing the air supply to enter the air supply line 124. Once the valves 200 are open for the middle calibration gas line, S415-S425 are repeated for the middle calibration gas to record the voltage of the high calibration point for the first set of replaceable orifices 116/119, e.g., corresponding to the middle calibration gas.

While, the valve 200 for the middle calibration gas is still open, the processor 150B controls the switchover valves 180 to switch to open towards the second set of replaceable orifices 116A/119A, e.g., allow flow to the second set of replaceable orifices. The processor 150B instructs the switchover valve controller 185 to open the switchover valve 180 to the second set of orifices 116A/119A, e.g., processor 150B issues a control pulse or signal to the switchover valve controller 185. Once the valves 180 switch, S415-S425 are repeated for the middle calibration gas to record the voltage of the low calibration point for the second set of replaceable orifices 116A/119A, e.g., corresponding to the middle calibration gas.

At S915, the processor 150B controls the valves 200 to close (for the middle calibration gas line) and the valve 205 to open for the exhaust line 169. In an aspect of the disclosure, the processor 150B transmits a pulse signal to the valve controller 113B. The valve controller 113B controls the valves 200/205 using air to close or open the respective valves. Once the valves 200 are closed and the valve 205 is open, the calibration gas (middle) is isolated from the sample/calibration gas line downstream of the valve system 112B. The middle calibration gas may vent via the exhaust line 169 through the exhaust port 170. Once, the valves 200 for the middle calibration gas line are closed, S435, S415-S425 are repeated for the high calibration gas (and line) to record the voltage of the high calibration point for the second set of replaceable orifices 116A/119A, e.g., corresponding to the high calibration gas.

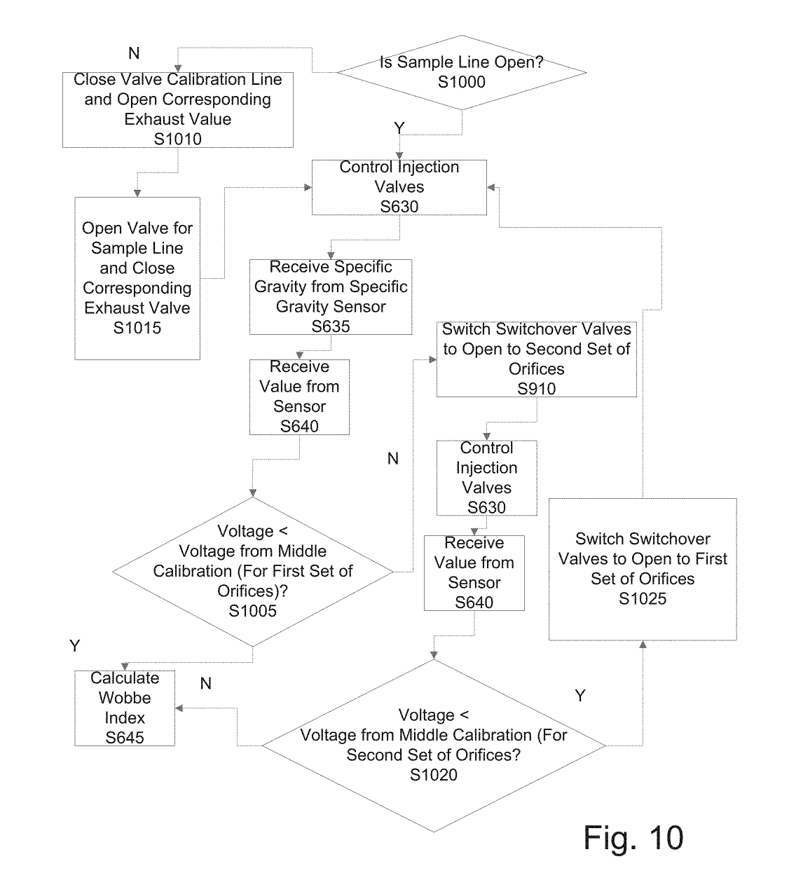

FIG. 10 depicts a flow chart for determining a wobbe index using apparatus 100B in accordance with aspect of the disclosure.

At S1000, the processor 150B determines whether the valves 200 for the sample line are open, e.g., is the sample gas coupled to the mixer 117. This determination may be made in accordance with any of the above-described techniques and will not be described again.

When the sample line is closed (“N” at S1000), the processor 150B controls the valves 200 for the opened calibration gas line to close and corresponding valve 205 to open to the exhaust line 169. At S1015, the processor 150B controls the valves 200 for the sample line to open and corresponding valve 205 to close (to the exhaust line 169). Subsequently, the process moves to S630-S640. The air supply valve 118 is open at this time. If at S1000, the sample line is open (“Y” at S1000), the process moves to S630-S640. S630-S640 were described above and will not be described again.

At S1005, the processor 150B compares the voltage level indicated by the sensor 135 (e.g., received) with the stored calibration points, e.g., low and high for the first set of replaceable orifices. If the voltage level is less than the high calibration point (“Y” at S1005), e.g., point generated from the middle calibration gas, the processor 150B uses the voltage level to calculate the wobbe index using equation 1. If the voltage level indicated by the sensor 135 (e.g., received), is greater than or equal to the high calibration point (“N” at S1005), the processor 150B controls the valves 180 to open toward the second set of replaceable orifices 116A/119A, e.g., switch, at S910. The processor 150B issues an instruction to the switchover valve controller 185 to cause the switching. S630 and S640 are repeated.

At S1020, the processor 150B compares the voltage level indicated by the sensor 135 (e.g., received) with the stored calibration points, e.g., low and high for the second set of replaceable orifices. If the voltage level is greater than the lower calibration point (“N” at S1020), e.g., point generated from the middle calibration gas, the processor 150 B uses the voltage level to calculate the wobbe index using equation 1.

On the other hand, if the voltage level is less than or equal to the low calibration point (“Y” at S1020), the processor 150B controls the valves 180 to open toward the first set of replaceable orifices 116/119, e.g., switch, at S1025. The processor 150B issues an instruction to the switchover valve controller 185 to cause the switching. The process returns to S630.

In accordance with aspects of the disclosure, features of the different apparatuses 100, 100A and 100B may be combined into an apparatus. For example, an apparatus may comprises a plurality of sample lines in combination with multiple sets of replaceable orifices. In accordance with aspects of the discloses any number of sets of orifices may be used and the disclosure is not limited to have two as depicted in FIG. 8.

The apparatuses 100, 100A and 100B may be a calorimeter.

The dashed lines in FIGS. 1, 5, and 8 represent control signals/instructions from the processor or data to the processor. The dashed lines in FIG. 2 represent control airflow from the valve controller 113/113A/113B.

The Processors 150, 150A and 150B includes at least one data storage device, such as, but not limited to, RAM, ROM and persistent storage (not shown in the figures). In an aspect of the disclosure, the Processor 150, 150A and 150B can be configured to execute one or more programs stored in a computer readable storage device including a program for supplying signals to the respect injection controllers 106, 107.

The computer readable storage device can be RAM, persistent storage or removable storage. A storage device is any piece of hardware that is capable of storing information, such as, for example without limitation, data, programs, instructions, program code, and/or other suitable information, either on a temporary basis and/or a permanent basis.

Various aspects of the present disclosure may be embodied as a program, software, or computer instructions embodied or stored in a computer or machine usable or readable medium, or a group of media which causes the computer or machine to perform the steps of the method when executed on the computer, processor, and/or machine. A program storage device readable by a machine, e.g., a computer readable medium, tangibly embodying a program of instructions executable by the machine to perform various functionalities and methods described in the present disclosure is also provided, e.g., a computer program product.

The computer readable medium could be a computer readable storage device or a computer readable signal medium. A computer readable storage device, may be, for example, a magnetic, optical, electronic, electromagnetic, infrared, or semiconductor system, apparatus, or device, or any suitable combination of the foregoing; however, the computer readable storage device is not limited to these examples except a computer readable storage device excludes computer readable signal medium. Additional examples of the computer readable storage device can include: a portable computer diskette, a hard disk, a magnetic storage device, a portable compact disc read-only memory (CD-ROM), a random access memory (RAM), a read-only memory (ROM), an erasable programmable read-only memory (EPROM or Flash memory), an optical storage device, or any appropriate combination of the foregoing; however, the computer readable storage device is also not limited to these examples. Any tangible medium that can contain, or store, a program for use by or in connection with an instruction execution system, apparatus, or device could be a computer readable storage device.

A computer readable signal medium may include a propagated data signal with computer readable program code embodied therein, such as, but not limited to, in baseband or as part of a carrier wave. A propagated signal may take any of a plurality of forms, including, but not limited to, electro-magnetic, optical, or any suitable combination thereof. A computer readable signal medium may be any computer readable medium (exclusive of computer readable storage device) that can communicate, propagate, or transport a program for use by or in connection with a system, apparatus, or device. Program code embodied on a computer readable signal medium may be transmitted using any appropriate medium, including but not limited to wireless, wired, optical fiber cable, RF, etc., or any suitable combination of the foregoing.

The term “Processor” as may be used in the present disclosure may include a variety of combinations of fixed and/or portable computer hardware, software, peripherals, and storage devices. The “Processor” may include a plurality of individual components that are networked or otherwise linked to perform collaboratively, or may include one or more stand-alone components.

The terminology used herein is for the purpose of describing particular embodiments only and is not intended to be limiting the scope of the disclosure and is not intended to be exhaustive. Many modifications and variations will be apparent to those of ordinary skill in the art without departing from the scope and spirit of the disclosure.

What is claimed is:

1. An apparatus comprising:
an air supply line for supplying air;
a first replaceable orifice connected to the air supply line; a process gas supply line for supplying gas under testing;
a plurality of calibration gas supply lines, each respectively supplying a different gas for calibration;
a valve system coupled to the plurality of calibration gas supply lines and the process gas supply line for selectively supply the gas under testing or one of the different gases for calibration to a common supply line;
a second replaceable orifice connected to the common supply line;
a mixer having a first input and a second input and a gas output, air from the first replaceable orifice is supplied to the first input and gas for calibration or gas under testing from the second replaceable orifice is supplied to the second input;
a first injection valve connected the air supply line and the first replaceable orifice configured to inject the air into the first replaceable orifice;
a second injection valve connected to the common supply line and the second replaceable orifice, the second injection valve configured to inject gas for calibration or gas under testing into the second replaceable orifice;
output gas line connected to the gas output, the output gas line also being connected to a heating element;
a sensor disposed near the heating element configured to sense property of output of the heating element and providing a signal indicating of the property;
a first controller configured to control the first injection valve at a preset frequency and a duty cycle;
a second controller configured to control the second injection valve at the preset frequency and the duty cycle, wherein the preset frequency and the duty cycle and dimensions of the output gas line are set to have a continuous gas flow into the heating element, wherein the signal from the sensor is used to determine a measure of a WOBBE index of the gas under testing.

2. The apparatus of claim 1, further comprising a casing having a first compartment, a second compartment and a third compartment, the first controller and second controller is disposed in the first compartment and the heating element and sensor being disposed in the second compartment.

3. The apparatus of claim 1, wherein the first controller and the second controller each comprise a solenoid.

4. The apparatus of claim 1, further comprising:
a first pressure regulator connected between a source of the air and the first replaceable orifice to control the pressure of said air into the first replaceable orifice; and
a second pressure regulator connected between a source of either the gas under testing or the gases for calibration and the second replaceable orifice to control the pressure of said gas under testing or gases for calibration into the second replaceable orifice.

5. The apparatus of claim 1, further comprising a processor configured to determine a measure of the WOBBE index based on the received signal from the sensor.

6. The apparatus of claim 1, wherein sensor is an oxygen sensor.

7. The apparatus of claim 1, wherein the mixer is a static mixer.

8. The apparatus of claim 1, further comprising a plurality of process gas supply line, the plurality of process gas supply line comprising the process gas supply line, each of the plurality of process gas supply line coupleable to a different gas under testing.

9. The apparatus of claim 8, further comprising a valve controller, the valve controller configured to open or close a respective valve in the valve system to selectively supply one of the different gases under testing to the common supply line under control of the processor.

10. The apparatus of claim 9, wherein the processor is configured to communicate with a distributed control system and receive an instruction from the distributed control system to cause one of the different gases under testing to be supplied to the common supply line.

11. The apparatus of claim 9, wherein the processor comprises a memory storing a time schedule for selectively supplying one of the different gases under testing to the common supply line, and configured to control the valve controller based on the time schedule.

12. The apparatus of claim 9, wherein the processor is configured to:
receive an instruction from a local user interface to selectively supply one of the different gases under testing to the common supply line, and
control the valve controller based on the instruction.

13. The apparatus of claim 1, further comprising:
a third replaceable orifice connected to the air supply line and a first switchover valve, the first switchover valve switching the air flow towards one of the first replaceable orifice and the third replaceable orifice;
a fourth replaceable orifice connected to the common supply line and a second switchover valve, the second switchover valve switching a flow of the gas under testing or one of the different gas for calibration towards one of the second replaceable orifice and the fourth replaceable orifice.

14. The apparatus of claim 13, wherein a diameter of the third replaceable orifice is greater than a diameter of the first replaceable orifice and wherein a diameter of the fourth replaceable orifice is smaller than a diameter of the second replaceable orifice.

15. The apparatus of claim 14, further comprising a switchover valve controller configured to switch the first switchover valve and the second switchover valve under controller of the processor.

16. The apparatus of claim 15, wherein when air flows towards the first replaceable orifice and the gas under testing flows towards the second replaceable orifice, the processor is configured to compare a level indicated by the signal from the sensor with a preset first threshold, and when the level indicated by the signal is greater than the preset first threshold, the processor is configured to control the switchover valve controller to cause the first switchover valve to allow air to flow towards the third replaceable orifice and cause the second switchover valve to allow the gas under testing to flow towards the fourth replaceable orifice.

17. The apparatus of claim 15, wherein when air flows towards the third replaceable orifice and the gas under testing flows towards the fourth replaceable orifice, the processor is configured to compare the level indicated by the signal from the sensor with a preset second threshold, and when the level indicated by the signal is less than the preset second threshold, the processor is configured to control the switchover valve controller to cause the first switchover valve to allow air to flow towards the first replaceable orifice and cause the second switchover valve to allow the gas under testing to flow towards the second replaceable orifice.

18. The apparatus of claim 16, wherein when air flows towards the third replaceable orifice and the gas under testing flows towards the fourth replaceable orifice, the processor is configured to compare the level indicated by the signal from the sensor with a preset second threshold, and when the level indicated by the signal is less than the preset second threshold, the processor is configured to control the switchover valve controller to cause the first switchover valve to allow air to flow towards the first replaceable orifice and cause the second switchover valve to allow the gas under testing to flow towards the second replaceable orifice.

19. The apparatus of claim 18, wherein the preset first threshold and the preset second threshold are set during calibration using a gas having a known energy, wherein the preset first threshold is determined when the gas having the known energy flows through the second orifice and air flows through the first orifice and the preset second threshold is determined when the gas having the known energy flows through the fourth orifice and air flows through the third orifice.

20. The apparatus of claim 13, further comprising a plurality of process gas supply line, the plurality of process gas supply line comprising the process gas supply line, each of the plurality of process gas supply line coupleable to a different gas under testing.
